(19) United States
(12) Patent Application Publication
(10) Pub. No.: US 2025/0249218 A1
(43) Pub. Date: Aug. 7, 2025

(54) MEDICAL DEVICES, SENSORS FOR MEDICAL DEVICES AND RELATED METHODS

(71) Applicant: XENTER, INC., Draper, UT (US)

(72) Inventor: F. Levent DEGERTEKIN, Draper, UT (US)

(21) Appl. No.: 18/855,202

(22) PCT Filed: Apr. 7, 2023
(86) PCT No.: PCT/US2023/017820

§ 371 Date: Oct. 8, 2024

Related U.S. Application Data
(60) Provisional application No. 63/328,842, filed on Apr. 8, 2022.

(51) Int. Cl. A61M 25/09; A61B 8/08; A61B 8/12; B41M 1/12; B41M 1/26; B41M 5/00

(52) U.S. Cl. CPC A61M 25/09; A61B 8/0891; A61B 8/12; B41M 1/12; B41M 1/26; B41M 5/0047; A61B 2562/04; A61B 2562/12; A61M 2025/09108; A61M 2205/0294

(57) ABSTRACT

Medical devices incorporating sensors, systems using such medical devices, methods of manufacture and methods of use are provided. In one, non-limiting example, such medical devices may include intraluminal devices, such as guidewires and/or catheters, which include various sensors for detecting, imaging, and/or measuring of one or more physiological parameters. In one embodiment a guidewire may comprise an elongated element and at least one sensor comprising a piezoelectric material having a curing temperature of approximately 100° C. or less and an acoustic impedance of approximately 14 MRayl or less.

### CROSS-REFERENCE TO RELATED APPLICATIONS

This application claims the benefit of U.S. Provisional Patent Application No. 63/328,842, entitled MEDICAL DEVICES, SENSORS FOR MEDICAL DEVICES AND METHODS OF MANUFACTURING, filed on Apr. 8, 2022, which is incorporated herein by reference in its entirety.

### BACKGROUND

The present disclosure relates generally to medical devices incorporating sensors. In one, non-limiting example, such medical devices may include intraluminal devices, such as guidewires and catheters, which include various sensors for detecting, imaging, and/or measuring of one or more physiological parameters.

Guidewire devices are often used to lead or to guide a catheter or other interventional devices to a targeted anatomical location within a patient's body. Typically, guidewires are passed into and through a patient's vasculature in order to reach the target location, which may be, for example, at or near the patient's heart or brain. Radiographic imaging is typically utilized to assist in navigating a guidewire to the targeted location. Guidewires are available with various outer diameter sizes. For example, widely utilized sizes include 0.010, 0.014, 0.016, 0.018, 0.024, and 0.035 inches in diameter, for example, though they may also be smaller or larger in diameter.

In many instances, a guidewire is placed within the body during the interventional procedure where it can be used to guide multiple catheters or other interventional devices to the targeted anatomical location. Once in place, a catheter can be used to aspirate clots or other occlusions, or to deliver drugs, stents, embolic devices, radiopaque dyes, or other devices or substances for treating the patient.

These types of interventional devices can include sensors located at the distal end in order to provide added functionality to the device. For example, intravascular ultrasound (IVUS) is an imaging technique that utilizes a catheter with an ultrasound imaging sensor attached to the distal end. Ultrasound is utilized to image within targeted vasculature (typically the coronary arteries). In another example, the sensors may include a physiological sensor configured to measure, for example, pressure, temperature, or flow within a vessel.

There are several challenges associated with using sensors with intraluminal devices. For example, such interventional devices involved have very limited space to work in given the stringent dimensional constraints involved. Moreover, integrating the sensors with the interventional device in a way that maintains effective functionality can be challenging.

There is an ongoing need for improved medical devices that effectively integrate sensors and can help provide data in a more efficient manner and/or provide data not previously obtainable in a practical manner.

### SUMMARY

Medical devices incorporating sensors, systems using such medical devices, methods of manufacture and methods of use are provided. In one, non-limiting example, such medical devices may include intraluminal devices, such as guidewires and/or catheters, which include various sensors for detecting, imaging, and/or measuring of one or more physiological parameters.

In one embodiment, a guidewire is provided that includes an elongated element and at least one sensor. The at least one sensor comprises a piezoelectric material having a curing temperature of approximately 100° C. or less (and optionally at least about 50° C., 60° C., 70° C., or 80° C.) and an acoustic impedance of approximately 14 MRayl or less (and optionally at least about 1.5 MRayl, 3 MRayl, 5 MRayl, 7.5 MRayl, or 10 MRayl).

In one embodiment, the at least one sensor includes a plurality of sensors disposed circumferentially about a portion of the elongated element.

In one embodiment, the plurality of sensors includes at least eight sensors.

In one embodiment, the plurality of sensors exhibit a pitch (i.e., circumferential distance from one sensor to the next) of approximately 125 microns (μm) or less.

In one embodiment, the plurality of sensors exhibit a pitch of approximately 62.5 microns (μm) or less.

In one embodiment, the plurality of sensors exhibit a pitch of approximately 31 microns (μm) or less.

In one embodiment, the elongated element comprises at least one of a core wire and a hypo tube and wherein the at least one sensor is formed directly on the core wire or the hypo tube.

In one embodiment, the at least one sensor is formed on a polymer film.

In one embodiment, the polymer film comprises polyimide.

In one embodiment, the polymer film is disposed on a core wire or a hypotube.

In another embodiment, a method is provided that includes providing an elongated body and positioning at least one sensor along a portion of the elongated body, including forming the at least one sensor of a piezoelectric material at a temperature of approximately 100° C. or less and an acoustic impedance of approximately 14 MRayl or less.

In one embodiment, the method further comprises positioning the sensor in a vessel and emitting an ultrasonic wave from the at least one sensor to determine a characteristic of the vessel.

In one embodiment, the method further comprises detecting a reflection of the emitted ultrasonic wave to determine the characteristic of the vessel.

In one embodiment, the characteristic of the vessel includes a size of a lumen of the vessel.

In one embodiment, forming the at least one sensor includes forming a plurality of sensors and circumferentially arranging the plurality of sensors about the portion of the elongated body.

In one embodiment, emitting an ultrasonic wave from the at least one sensor includes emitting an ultrasonic wave from each of the plurality of sensors to obtain an ultrasonic image of the vessel.

In one embodiment, forming the at least one sensor of a piezoelectric material includes screen printing the piezoelectric material onto a substrate.

In one embodiment, forming the at least one sensor of a piezoelectric material includes jet printing the piezoelectric material onto a substrate.

In one embodiment, the method comprises forming the at least one sensor on a polymer film and then disposing the polymer film on the elongated body.

In one embodiment, forming the at least one sensor of a piezoelectric material includes forming the at least one sensor directly on at least one of a core wire or a hypo tube of an elongated body.

Components, features, elements, steps or acts of one embodiment may be combined with components, features, elements, steps or acts of other embodiments without limitation.

### BRIEF DESCRIPTION OF THE DRAWINGS

The foregoing and other advantages of various embodiments of the disclosure will become apparent upon reading the following detailed description and upon reference to the drawings in which:

FIG. 1 illustrates a guidewire system according to an embodiment of the present disclosure;

FIG. 2 is a side view of a distal portion of a guidewire according to one embodiment of the present disclosure;

FIG. 3 is a perspective view of a distal portion of a guidewire according to one embodiment of the present disclosure;

FIGS. 4A-4C depict various acts in a manufacturing process associated with a guidewire in accordance with an embodiment of the present disclosure;

FIGS. 5A-5D illustrates the use of the guidewire in accordance with an embodiment of the disclosure;

FIGS. 6A-6C are graphs showing responses of a guidewire configured according to an embodiment of the present disclosure.

### DETAILED DESCRIPTION

Various embodiments described herein are directed toward medical devices, systems, and related methods, including the incorporation of sensors into medical devices, the manufacture of such sensors and devices, and the use of such sensors and devices.

In some embodiments, devices associated with cardiovascular, neurovascular, and endovascular procedures are provided having sensors integrated therewith. For example, guidewires or catheters may include sensors integrated into the structure for detecting or measuring physiological data (e.g., pressure, flow rate, etc.) or to provide imaging data, and which provide that data in real time during an associated procedure.

In some embodiments, the sensors, transducers, or electronic elements incorporated into the device may be formed through manufacturing techniques such as a screen printing, aerosol jet printing technologies, 3D printing, stereolithography, microlithography, or combinations of such manufacturing techniques and processes.

Some non-limiting examples of medical devices that may incorporate such sensors include those described in: U.S. patent application Ser. No. 17/205,964, entitled “Guidewire for Imaging and Measurement of Pressure and Other Physiological Parameters” and filed on Mar. 18, 2021; U.S. patent application Ser. No. 17/205,854, entitled “Catheter for Imaging and Measurement of Pressure and Other Physiological Parameters” and filed on Mar. 19, 2021; U.S. patent application Ser. No. 17/205,754, entitled “Operatively Coupled Data and Power Transfer Device for Medical Guidewires and Catheters with Sensor” and filed on Mar. 18, 2021; U.S. patent application Ser. No. 17/205,614, entitled “Signal Conducting Device for Concurrent Power and Data Transfer to and From Un-wired Sensors Attached to a Medical Device” and filed on Mar. 18, 2021; U.S. patent application Ser. No. 17/979,629, entitled “Data and Power Transfer Devices for Use with Medical Devices and Related Methods” and filed Nov. 2, 2022; U.S. patent application Ser. No. 18/114,141, entitled “Medical Devices, Systems, and Methods Incorporating the Same” and filed Feb. 24, 2023; and U.S. Provisional Patent Application No. 63/394,591, entitled “Medical Devices, Systems, and Methods Including Power and Data Transfer” and filed on Aug. 2, 2022, 20 the disclosures of which are each incorporated by reference herein in their entireties.

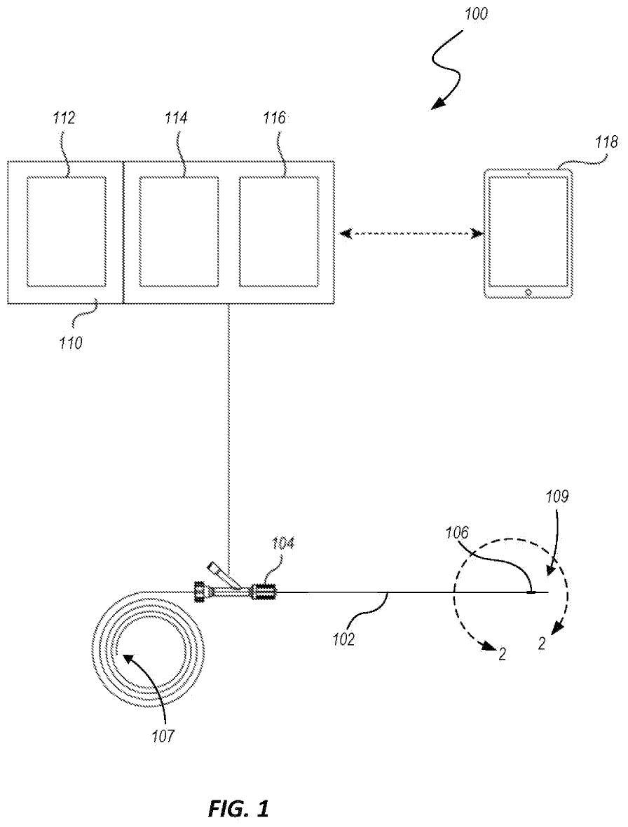

Referring to FIG. 1, a guidewire system 100 is illustrated according to an embodiment of the present disclosure. As shown, the guidewire system 100 includes a wire 102 (which is an example of an “elongated body” or “elongated element” as used herein), a proximal device 104, one or more sensors 106 associated with the wire 102, a control unit 110 (shown enlarged and in schematic form) that includes a power source 112, data signal processor 114, and optionally a transmitter 116, and an external device 118 (e.g., a stationary or handheld display, tablet computer, or other input and/or output device) which may be in wired or wireless communication with the transmitter 116.

The proximal device 104 (which, in some embodiments may be associated with proximal portion and/or the intermediate portion, as defined below) may be configured as a data and/or power transfer device. For example, the proximal device 104 may be configured for electrical coupling with the wire 102 to provide power to sensors or other electronics associated with the wire and to receive various signals provided by the sensors or other electronic components. Such electrical coupling between the proximal device 104 and the wire 102 may be accomplished via conductive connection or via an electronic field (e.g., through a capacitive coupling). In some embodiments, the proximal device may include a hemostatic valve.

The data signal processor 114 may be configured to receive sensor data signals, sent through the wire 102, from one or more sensors 106 (which may be located and oriented in specific patterns as individual sensors, or may be configured as identifiable sensor arrays) associated with the wire 102. The power source 112 may be configured to transmit power through the wire 102 to power the one or more sensors 106 and/or other components of the wire 102. The power source 112 may include an on-board power source, such as a battery or battery pack, and/or may include a wired connection to an outside power source.

The one or more sensors 106 may be located at any suitable position on the wire 102 but will typically be disposed along the distal section which is expected to reach the targeted anatomy. As used herein, the “distal section” or “distal portion” refers to the distal-most 30 cm of the device, the distal-most 20 cm of the device, the distal-most 15 cm of the device, the distal-most 10 cm of the device, or to a range using any two of the foregoing values as endpoints. In some embodiments, the “intermediate section” may be considered as roughly the middle third of the device, and the “proximal section” or “proximal portion” may be considered as roughly the proximal third of the device.

In one embodiment, the guidewire system 100 is configured to send power to the one or more sensors 106 through individual traces or other electrical conductors, and data signals from the one or more sensors 106 may be transmitted via separate transmission structures or schemes. For example, data may be transmitted by additional traces or electrical conductors, by optical transmission, or by wireless transmission. In some embodiments, such transmission structures (e.g., traces) may be formed on/in, or otherwise integrated with, the wire 102 using additive manufacturing techniques.

In another embodiment, the guidewire system 100 is configured to send power and data signals through the actual wire 102 itself rather than through traces or through separate, discrete electrical conductors. In some embodiments, multiple power and/or data signals (e.g., data signals from multiple sensors) can be sent through the wire 102 simultaneously. Power and/or data signals can also be sent in a “continuous” fashion. That is, the power and/or data signals can have a sufficiently high sampling rate such that the information is provided to the user within time frames that are practically “real-time.” Nonlimiting examples of managing power and/or data signals, along with associated components and systems, as set forth in the previously incorporated U.S.

### PATENT DOCUMENTS

The wire 102 of the guidewire system 100 is configured for insertion into the body of a subject. The subject is typically a human, but in other implementations may be a non-human mammal or even non-mammalian animal. Any suitable route of administration may be utilized, depending on particular preferences and/or application needs. Common routes include femoral, radial, and jugular, but the guidewire system 100 may utilize other access routes as needed.

The wire 102 has a proximal end 107 and a distal end 109. The length of the wire 102 may vary according to particular application needs and targeted anatomical area. As an example, the wire 102 may have an overall length from proximal end 107 to distal end 109 of about 50 cm to about 350 cm, more commonly about 200 cm, depending on particular application needs and/or particular anatomical targets. The wire 102 may have a size such that the outer diameter (e.g., after application of other outer members) is about 0.008 inches to about 0.040 inches, though larger or smaller sizes may also be utilized depending on particular application needs. For example, particular embodiments may have outer diameter sizes corresponding to standard guidewire sizes such as 0.010 inches, 0.014 inches, 0.016 inches, 0.018 inches, 0.024 inches, 0.035 inches, 0.038 inches, a size range with any combination of the foregoing as endpoints, or other such sizes common to guidewire devices. The wire 102 may be formed from stainless steel or other metal or alloy having similar appropriate properties. In some embodiments, the wire 102 may be formed of or may comprise a conductive material of appropriate mechanical properties.

Given the fact that guidewires inherently involve strict dimensional and performance (e.g., torqueability, bending, pushability, stiffness, trackability, etc.) limitations and have limited space to work in, the ability to reduce or eliminate extraneous components frees up limited space and allows greater design flexibility. Additionally, the ability to integrate various components, such as sensors or other electronic components, also provides a more feature-rich, functional, and durable device. The integration of sensors 106 or other electrical elements or components with the wire 102 according to embodiments described herein may enable the production of a wire 102 having desired size and handling characteristics while also providing enhanced data capture and other functionality.

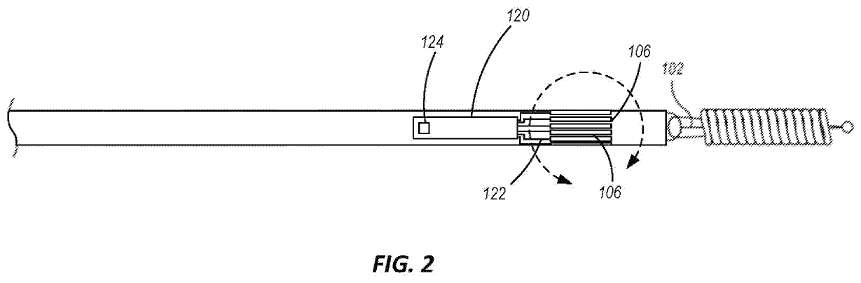

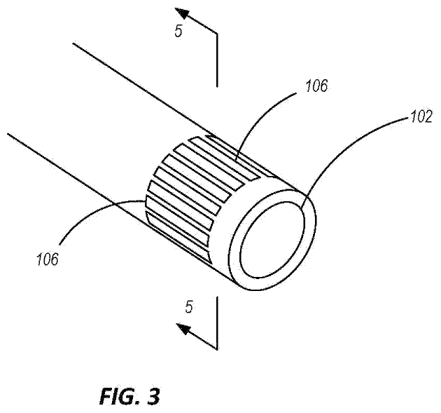

Referring to FIGS. 2 and 3, one or more sensors 106 of the guidewire system 100 may be arranged circumferentially about the guidewire 102 in an array, with each sensor element 106 being in electrical communication with a processor 120 (shown schematically in FIG. 2) by way of a plurality of traces 122 or other conductors. In one embodiment, the processor 120 may include an application specific integrated circuit (ASIC). The processor 120 may be configured to control the operation of the sensors 106 such as described in further detail below. In some embodiments, the processor 120 may include a chip having one or more additional sensors 124 integrated therewith (e.g., pressures sensors, flow sensors, temperature sensors, etc.).

It is noted that, while generally referred to as a “sensor” in discussing various embodiments throughout, “sensors” and/or “sensor components” (e.g., sensor 106) may comprise or otherwise be associated with transducers or other components and may be configured as input devices, output devices, or both. In other words, sensor components described herein may include or be a part of transducers that are capable of transmitting and/or receiving a specified type of signal. For example, such sensors may include ultrasound transducers, optical emitters (e.g., light emitting diodes (LEDs)), optical sensors (e.g., photo diodes) and the like.

In one particular embodiment, the sensors 106 may be configured as ultrasonic transducers formed of a piezoelectric material. In one embodiment, the piezoelectric material may be applied directly to the guidewire 102 (e.g., to a core wire, a hypo-tube, or to a housing structure coupled with a core wire or a hypo tube). In another embodiment, the piezoelectric material may be applied to a film or coating placed on the guidewire 102. For example, the piezoelectric material may be applied to (and, thus, forming a piezo type ultrasonic transducer on) a tube structure (e.g., polyimide) or other structure that is positioned over a portion of the guidewire 102. In another embodiment, the piezoelectric material may be applied to a film (e.g., polyimide) while the film is in a flat state, the film being subsequently wrapped or otherwise conformally applied to a surface of the guidewire 102. Example films include polyimides, polyamide-imides (PAIs), polybenzimidazoles (PBIs), polyphenylene oxides (PPOs), polyetherimides (PEIs), and combinations thereof.

In one example, the piezoelectric material may include PiezoPaint™, available from Meggitt PLC of the United Kingdom. In one example, the piezoelectric material may exhibit one or more of the following electrical properties: a relative dielectric permittivity of approximately 80-100 at 1 kHz; a dielectric dissipation factor of approximately 2.5 to 3.5 10⁻² at 1 kHz; a Curie temperature (ceramic phase) of 330° C.; or a recommended working range of 80° C. In one example, the piezoelectric material may exhibit one or more of the following electromechanical properties: a coupling factor, thickness of approximately 8.2%; a piezoelectric charge coefficient d₃₃ of about 45 pC/N; a piezoelectric charge coefficient d₃₁ of about 15 pC/N; or a frequency constant, thickness of 1410 Hz m. In one embodiment, the piezoelectric material may exhibit one or more of the following mechanical characteristics: an acoustic impedance of approximately 14 MRayl (1 MRayl equaling 106 kilogram/(second*meter²)) or less; density of 5.0 g/cm³; Young's modulus of 29 GPa; or Poisson's ration of 0.3. The piezoelectric material may also exhibit a low curing temperature of less than approximately 100° C. Such characteristics and properties are provided as examples and are not to be considered limiting.

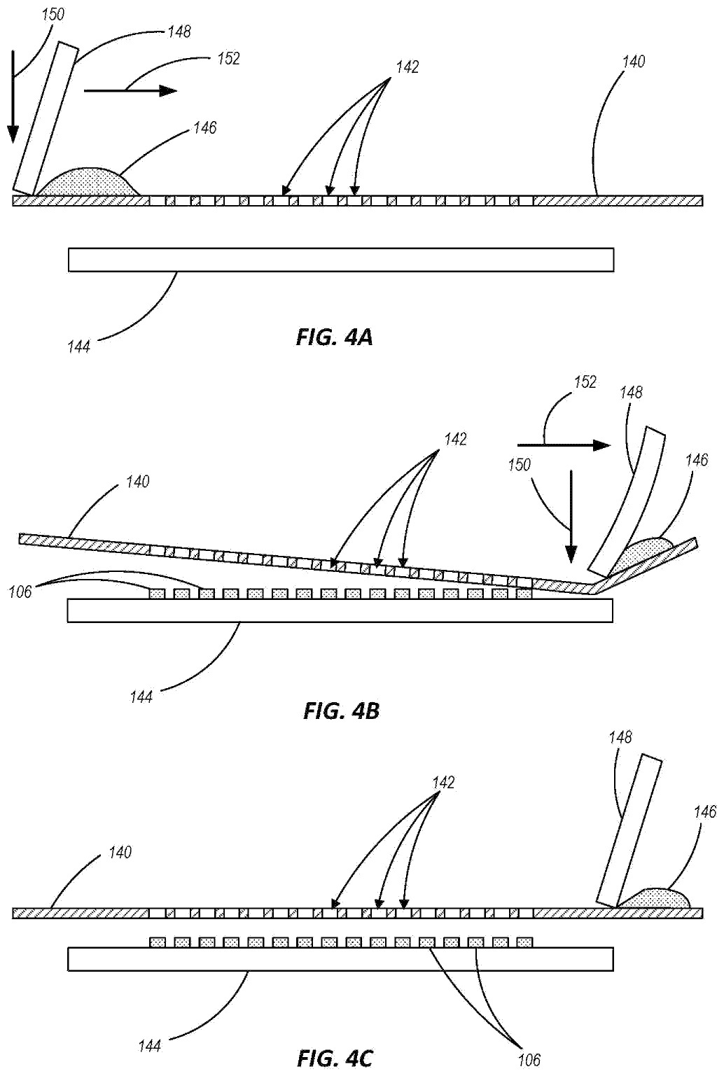

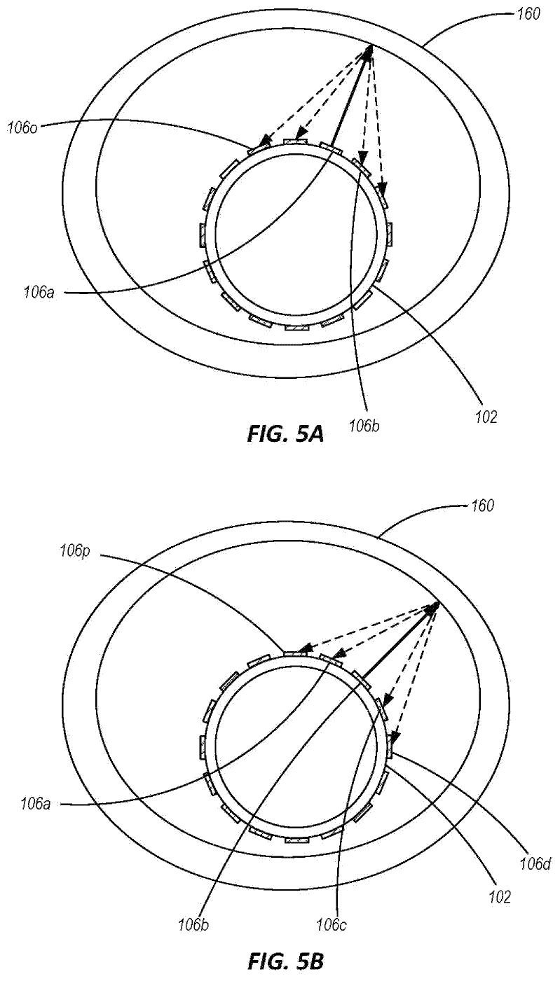

Referring to FIGS. 4A-4C, a method of forming sensors 106 on a substrate (e.g., on a polyimide film) is illustrated according to one embodiment of the present disclosure. As shown in FIG. 4A, a screen 140 having various openings 142 formed therein is positioned about a substrate 144. The substrate 144 may include a film (e.g., polyimide film) such as discussed above. In other embodiments, the substrate 144 may be formed as a non-planar geometric structure (e.g., a tube). In some embodiments, the substrate 144 may include a component of the guidewire 102 (e.g., a core wire, a hypo tube, a housing structure).

The openings 142 of the screen may be configured to be representative of a desired shape that the sensors to be formed. For example, the screen 140 is shown in cross-section so that a square or rectangular opening 142 is depicted. However, that opening may extend a desired length into (and/or out of) the page to provide or define a “length” of the resulting sensor. Of course, the sensors (and thus the openings) may exhibit other shapes or geometries.

A mass of piezoelectric material 146 is positioned on an upper portion of the screen 140 and a squeegee 148 is used to distribute the piezoelectric material into the openings and onto a desired location on the substrate 144. For example, a downward force is applied to the screen 140 by the squeegee 148 as indicated by directional arrow 150, causing at least a portion of the screen 140 to come into contact with the substrate as the squeegee 148 is drug across the screen 140. While the downward force is being applied by the squeegee 148, the squeegee 148 is also dragged or swept across the screen 140, as indicated by directional arrow 152, causing piezoelectric material 146 to be pressed or packed into the openings 142 to form the physical structure of the sensors 106 on the substrate as illustrated in FIGS. 4B and 4C. The resulting structures may then be cured to provide a structure that may be used as an ultrasonic transducer which may both emit and detect ultrasonic waves.

In some embodiments, the substrate 144 may be rolled onto a core wire or other structure as desired. In some embodiments, a filler material (e.g., a polyimide material) may be disposed between adjacent sensors 106 creating a substantially continuous surface between adjacent sensors. In some embodiments, a coating material may be disposed over the sensors. The formed sensors 106 may be coupled to other electrical components by way of traces or other conductors as previously discussed.

In other embodiments, other manufacturing processes and techniques may be used, such as noted above, including various spray, printing, and additive manufacturing techniques.

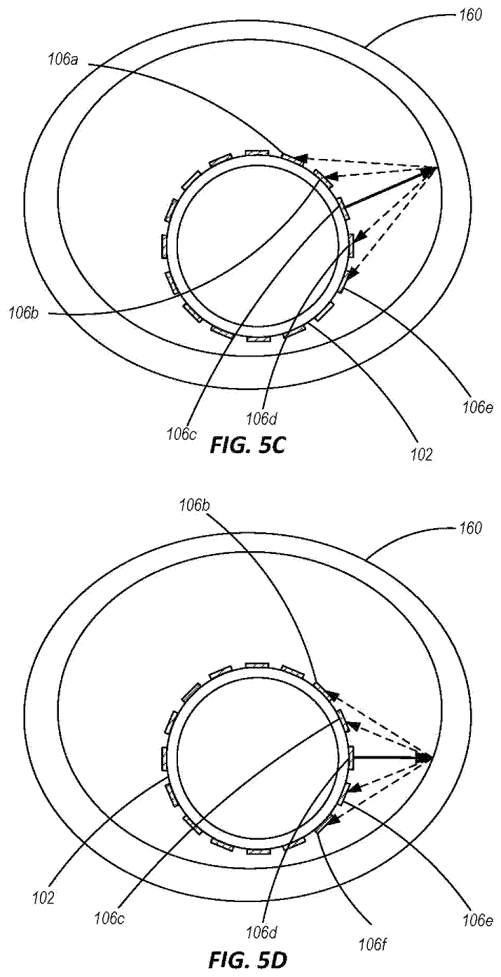

Referring to FIGS. 5A-5D, an example of the guidewire 102 being used as an imaging device is illustrated. As shown in FIG. 5A, a cross-section of the guidewire 102 is depicted as indicated by the section 5-5 in FIG. 3 and while it is positioned within a vessel 160. In the embodiment shown in FIGS. 5A-5D, sixteen individual sensors (106a-106p) are circumferentially positioned on the guidewire 102, though other embodiments may include a greater or lesser number of sensors. For example, one embodiment may include eight sensors having a pitch of 125 microns (μm). In another embodiment, sixteen sensors may be used having a 62.5 μm pitch. In yet another embodiment, thirty-two sensors may be used having a 31 μm pitch. In other embodiments, the pitch may be greater or less than the example values provided above. In various embodiments, the number of sensors and the pitch of the sensors may depend at least in part on the desired size (e.g., diameter) of the guidewire. In some instances, the number, size, and/or pitch of the sensors may depend, in part, on desired resolution of the resulting image to be obtained by the device.

In an embodiment having sixteen sensors 106, each sensor covers 22.5 degrees. If the width is smaller than the emitted wavelength divided by two (e.g., 75 μm at 20 MHz), then multiple elements will see each other's reflections. For example, a first sensor 106a (being configured as an ultrasonic transducer) may emit an ultrasonic wave in a substantially normal (perpendicular) direction relative to its radial surface which will be reflected (at least in part) from the wall of the vessel 160. The reflections will return and be detected by adjacent sensors (e.g., sensors 106b, 106c, 1060 and 106p). It is noted that the emitted ultrasonic waves are indicated by a solid line while reflected waves are indicated by dashed lines.

Knowing the geometrical arrangement of the sensors 106, the properties of the ultrasonic waves and response time of the reflections (e.g., the amount of time elapsed from emission of an ultrasonic wave to its detection of the reflection by a specific sensor), a distance of a particular point on the vessel from the receiving sensors (e.g., 106b, 106c, 1060 and 106p) may be determined. In some other cases, the same emitting sensor 106a can also be used as the receiver sensor in a monostatic fashion. This process may be repeated using different sensors as the emitting sensor. For example, FIG. 5B shows the emitting sensor as sensor 106b, FIG. 5C shows the emitting sensor as sensor 106c and FIG. 5D shows the emitting sensor as sensor 106d. In each case, the sensors located adjacent to the emitting sensor (e.g., one or more sensors on each side of the emitting sensor) can become detecting sensors with the process repeated as necessary such that all of the sensors (106a-106p) may be the emitting sensor. This entire process may happen numerous times each second such that the entire circumference of a vessel may be imaged while the guidewire 102 is pulled back or is otherwise displaced through a vessel.

In addition to the timing of the sensors for distance determination, the amplitude of the reflections can provide information on the local reflectivity of the vessel wall. For example, if there is calcification on the vessel wall, the reflected signal can be stronger (higher in amplitude) as compared to a region of the vessel lumen without calcification. Therefore, by recording and processing the reflected signals, information on the disease state of the blood vessel, for example the calcification arc over the lumen, can also be obtained. Using the pullback method for measurement, the length of the calcification can also be measured.

While in some embodiments, due to the size of the sensors 106 on the guidewire (also considering the intended size of the resulting guidewire device), the materials used to form the sensors 106, and other relevant factors, the resolution of a resulting ultrasonic image may be lower than that obtained by, for example, a larger IVUS catheter using pMUT or cMUT imaging transducers. However, even a reduced resolution image may provide benefit when produced by a smaller, less intrusive device such as a guidewire. For example, use of a guidewire device as disclosed herein enables ultrasonic imaging and/or lumen sizing information without the need for subsequent catheterization, enabling shorter and simpler procedures in at least some cases. Additionally, even if resolution is reduced relative to conventional IVUS catheter procedures, the sensors 106 can provide lumen sizing of a particular vessel. Thus, a reduced area or a necked section in a vessel may provide indications of stenosis or other anomalies requiring treatment.

At least some embodiments of disclosed guidewire devices are smaller than conventional IVUS catheters. For example, while typical IVUS catheters are 2.6 Fr to 3.5 Fr (0.034 inches to 0.046 inches), guidewire devices disclosed herein may have a size less than 0.034 inches, such as 0.028 inches or less, 0.024 inches or less, 0.018 inches or less, or 0.014 inches or less, including sizes down to about 0.010 inches. Accordingly, guidewire devices disclosed herein enable imaging and/or lumen sizing using intravascular device sizes smaller than used in conventional IVUS procedures.

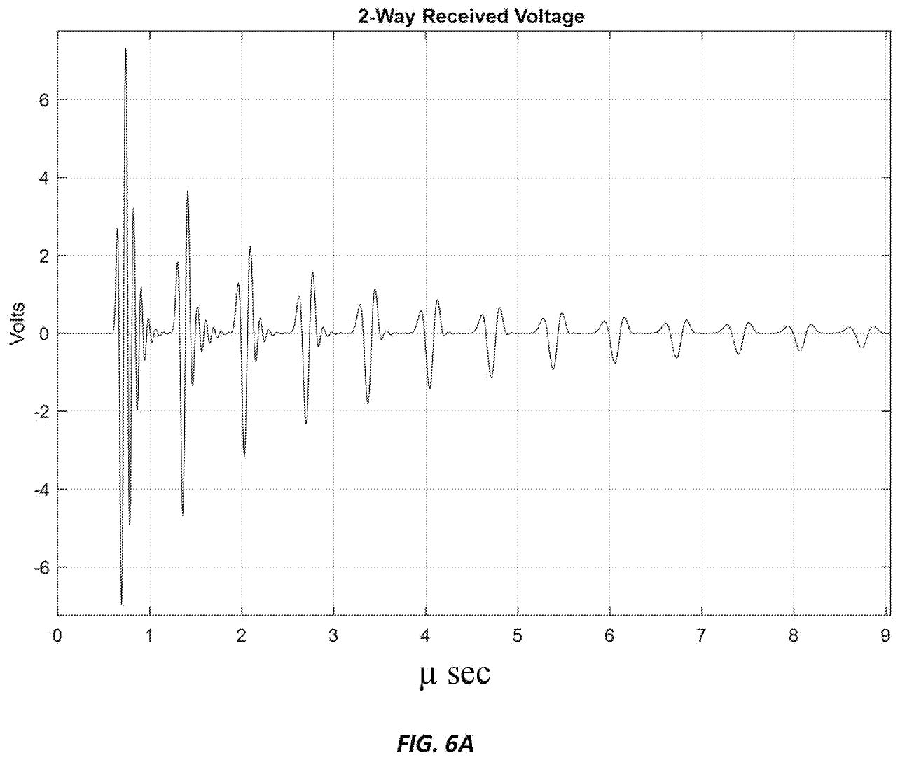

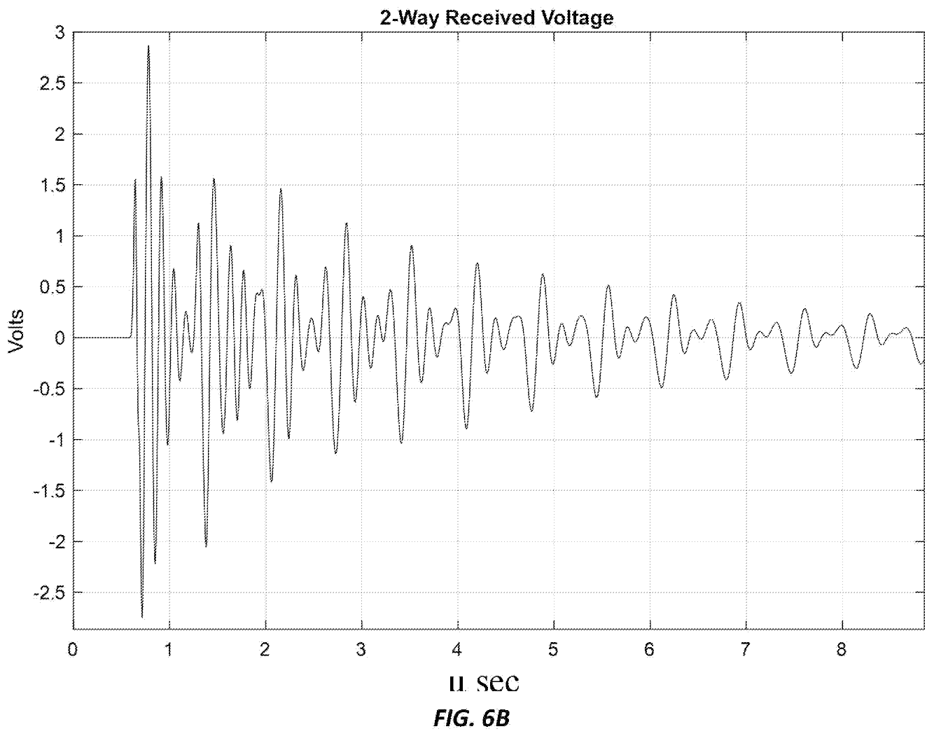

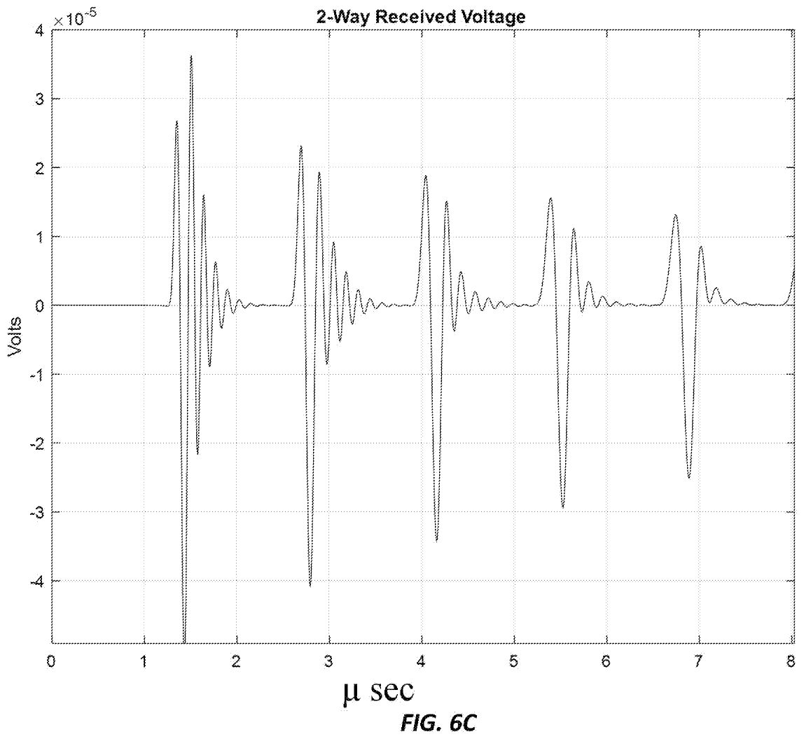

Referring briefly to FIGS. 6A through 6C, several graphs are provided showing the response of sensors formed according to the embodiment described with respect to FIGS. 4A-4C. The graphs show a reflection detected by the ultrasonic sensors disposed in blood and positioned a known distance from an interface (simulating a vessel wall). The sensors were formed to exhibit a 0.1 mm² area (i.e., 0.1 mm×1.0 mm) and were 30 μm thick. The sensors were formed on a polymer material comprising polyimide which was disposed on a core material comprising steel (e.g., simulating a core wire or a hypo tube of a guidewire). FIG. 6A represents response times where the sensors were formed on a 5 μm thick polymer material and positioned 0.5 mm away from the reflecting interface; FIG. 6B represents response times where the sensors were form on a 15 μm thick polymer material and positioned 0.5 mm away from the reflecting interface; and FIG. 6C represents response times where the sensors were form on a 30 μm thick polymer material and positioned 1 mm from the reflecting interface. The units of the vertical or Y-axis are Volts, while the units of the horizontal or X-axis are microseconds.

While the example embodiments have been set forth in terms of a guidewire, it is noted that various aspects of the disclosure may also be implemented with other devices such as catheters or implantable devices and that the present disclosure should not be viewed as being limited to guidewires.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for ease of explanation only and are not meant to be limiting.

Numerical values disclosed herein may optionally be modified by adding the term “about” or its synonyms. When the terms “about,” “approximately,” “substantially,” “roughly,” or the like are used in conjunction with a stated amount, value, or condition, it may be taken to mean an amount, value or condition that deviates by less than 15%, less than 10%, less than 5%, less than 1%, less than 0.1%, or less than 0.01% of the stated amount, value, or condition.

While the described embodiments may be susceptible to various modifications and alternative forms, specific embodiments have been shown by way of example in the drawings and have been described in detail herein. However, the invention is not intended to be limited to the particular forms disclosed and it is noted that components, features, elements, steps or acts of one embodiment may be combined with components, features, elements, steps or acts of other embodiments without limitation. Rather, the invention includes all modifications, equivalents, and alternatives falling within the spirit and scope defined by the following appended claims.

1. A guidewire comprising:
an elongated element;
at least one sensor comprising a piezoelectric material having a curing temperature of approximately 100° C. or less and an acoustic impedance of approximately 14 MRayl or less.

2. The guidewire of claim 1, wherein the at least one sensor includes a plurality of sensors disposed circumferentially about a portion of the elongated element.

3. The guidewire of claim 2, wherein the plurality of sensors includes at least eight sensors.

4. The guidewire of claim 2, wherein the plurality of sensors exhibit a pitch of approximately 125 microns (μm) or less.

5. The guidewire of claim 2, wherein the plurality of sensors exhibit a pitch of approximately 62.5 microns (μm) or less.

6. The guidewire of claim 2, wherein the plurality of sensors exhibit a pitch of approximately 31 microns (μm) or less.

7. The guidewire of claim 1, wherein the elongated element comprises at least one of a core wire or a hypo tube and wherein the at least one sensor is formed directly on the core wire or the hypo tube.

8. The guidewire of claim 1, wherein the at least one sensor is formed on a polymer film.

9. The guidewire of claim 9, wherein the polymer film comprises polyimide.

10. The guidewire of claim 9, wherein the polymer film is disposed on a core wire or a hypotube.

11. The guidewire of claim 1, wherein the elongated element of the guidewire has an outer diameter less than 0.034 inches.

12. A method comprising:
providing an elongated body;
positioning at least one sensor along a portion of the elongated body including forming the at least one sensor of a piezoelectric material at a temperature of approximately 100° C. or less and an acoustic impedance of approximately 14 MRayl or less.

13. The method of claim 12, further comprising:
positioning the sensor in a vessel; and
emitting an ultrasonic wave from the at least one sensor to determine a characteristic of the vessel.

14. The method according to claim 13, further comprising detecting a reflection of the emitted ultrasonic wave to determine the characteristic of the vessel.

15. The method according to claim 14, wherein the characteristic of the vessel includes a size of a lumen of the vessel.

16. The method according to claim 13, wherein forming the at least one sensor includes forming a plurality of sensors and circumferentially arranging the plurality of sensors about the portion of the elongated body.

17. The method according to claim 16, wherein emitting an ultrasonic wave from the at least one sensor includes emitting an ultrasonic wave from each of the plurality of sensors to obtain an ultrasonic image of the vessel.

18. The method according to claim 12, wherein forming the at least one sensor of a piezoelectric material includes screen printing the piezoelectric material onto a substrate.

19. The method according to claim 12, wherein forming the at least one sensor of a piezoelectric material includes jet printing the piezoelectric material onto a substrate.

20. The method according to claim 12, further comprising forming the at least one sensor on a polymer film and then disposing the polymer film on the elongated body.

21. The method according to claim 12, wherein forming the at least one sensor of a piezoelectric material includes forming the at least one sensor directly on at least one of a core wire or a hypo tube of an elongated body.

22. The method according to claim 13, wherein the elongated body comprises a wire of a guidewire device, and wherein the characteristic of the vessel is determined without passing a catheter into the vessel.
